(19) United States
(12) Patent Application Publication
(10) Pub. No.: US 2009/0306050 A1
(43) Pub. Date: Dec. 10, 2009

(54) TREATMENT AND PREVENTION OF DEPRESSION WITH PAIN, DEPRESSION SECONDARY TO PAIN, AND OF NEUROPATHIC PAIN

(76) Inventor: Timothy Dinan, Cork (IE)

Correspondence Address: ARNOLD & PORTER LLP;ATTN: IP DOCKETING DEPT., 555 TWELFTH STREET, N.W., WASHINGTON, DC, 20004-1206, US

(21) Appl. No.: 12/278,223

(22) PCT Filed: Feb. 1, 2007
(86) PCT No.: PCT/IB07/00248

§ 371 Date: Dec. 11, 2008

Related U.S. Application Data
(60) Provisional application No. 60/765,245, filed on Feb. 3, 2006.

(51) Int. Cl. A61K 31/55; A61P 25/00

(52) U.S. Cl. 514/217

(57) ABSTRACT

In accordance with the present invention, it has been discovered that compounds exhibiting activity as a potent noradrenaline reuptake inhibitor (e.g., a NA: 5HT ratio of greater than or equal to about 1000:1), and activity at the dopamine D2 receptor sites (e.g., lofepramine) are effective in the treatment and prevention of various diseases and disorders associated with noradrenaline reuptake, such as pain predominant-type depression, depression secondary to chronic or neuropathic pain, and neuropathic pain itself.

### CROSS-REFERENCES TO RELATED APPLICATIONS

This application claims the benefit of the filing date of U.S. Provisional Patent Application No. 60/765,245, filed Feb. 3, 2006, the contents of which is herein incorporated by reference in its entirety. This application is also related to PCT/IB2005/002925, filed Sep. 30, 2005, the contents of which is herein incorporated by reference in its entirety.

### BACKGROUND OF THE INVENTION

### A. Depression With Painful Symptoms

Depression is diagnosed in accordance with the Diagnostic and Statistical Manual (DSM-IV) criteria. This rating scale requires that the patient has experienced a set number from a panel of symptoms describing depressed affect, suicidal thoughts, anhedonia and loss of energy or insomnia.

The DSM criteria for depression have been developed over a number of years and the current criteria have been developed by modification of DSM-III. Hence it is recognised that future refinements of these criteria in the future will produce a new definition of what it means to be depressed.

In recent years much attention has been given to the area of painful symptoms in depression although these are not recognised at this time as a diagnostic criterion for depression. It is evident that a significant number of depressed patients present with such so called “somatization” which may involve various aches and pains as well as GI disturbances. Such painful symptoms are not present in all depressed patients. However in some patients that may be a very predominant or the predominant symptom. There is some evidence that such symptoms play a more important role in some populations such as Latin American patients.

It is evident that there is a substantial but as yet poorly undefined sub-group of depressed patients whose disease manifests itself predominately in somatization and that such sub-group is in need of medicines which can effectively treat both the physical and affective symptoms either sequentially or simultaneously. We refer to this sub-group as “pain predominant type depression”.

The class of drugs known as Serotonin Noradrenalin Reuptake Inhibitors which includes venlafaxine, duloxetine and others is known to be effective in treating painful symptoms in depression. A number of authors have indicated that drugs causing reuptake inhibition of both monoamines i.e. serotonin and noradrenaline is the optimum approach. Thus, Wise T N et al. (Management of painful physical symptoms associated with depression and mood disorders. CNS Spectr, 2005 December; 10(12 Suppl 19): 1-13) states “clinical evidence indicates that dual acting agents may have an advantage in modulating pain over those agents that increase either serotonin or noradrenaline alone.”

A number of drugs in this SNRI class are being promoted as a solution to “depression with pain” most especially the new SNRI drug from Eli Lilly termed duloxetine. Despite the foregoing there remains a need to find other new drugs which are effective in this condition.

### B. Depression Secondary to Pain

In addition to the matter of depression manifesting itself through physical symptoms there is also the matter of chronic or neuropathic pain which induces depression in subjects due to its chronic nature. In patients with chronic pain referred for evaluation to comprehensive pain programmes, 8-50% have been reported to have current major depression (Smith G R. The epidemiology and treatment of depression when it co-exists with somatoform disorders somatization or pain. Gen Hosp Psychiatry 1992, 14: 265-272). In another analysis of 1,016 HMO members, the prevalence of depression was 12% in individuals with 3 or more pain complaints (Dworkin S F et al. Multiple pains and psychiatric disturbance: an epidemiological investigation. Arch Gen Psychiatry 1990, 47: 239-244).

The most serious consequences of major depression are suicide and increased rates of suicidal ideation and suicide completion have been reported by patients suffering from chronic pain conditions. (Magni et al. Suicidality in chronic abdominal pain; an analysis of the Hispanic Health and Nutrition Examination Survey (HHANES). Pain, 1998, 76: 137-144)

In a comparison of measures of emotional distress, self-reported depressive symptoms, and the presence of major depression in 211 patients in a pain clinic study, major depression was significantly related to self-reported disability and negative thoughts about pain. (Geisser M E at al. Negative affect, self report of depressive symptoms and clinical depression: relation to the experience of chronic pain. Clin J Pain 2000, 16: 110-120). Oncology patients with concomitant pain and depression were significantly more likely to request assistance in committing suicide as well as actively taking steps to commit suicide. In contrast those with pain in the absence of depression were unlikely to request the intervention of euthanasia and physician-assisted suicide (Emanuel et al. Euthanasia and physician-assisted suicide; attitudes and experiences of oncology patients, oncologists and the public. Lancet 1996, 347: 1810)

It is evident that among depressed patients there is also a sub-group of patients whose depression arises secondary to chronic pain. Such patients may have their depression effectively treated by for example use of SSRI antidepressants and may have their pain or disease symptoms treated using other drugs. Nevertheless there remains a need for new drugs and methods to treat these patients who experience significant levels of morbidity and mortality.

### C. Neuropathic Pain

Neuropathic pain is an important area of unmet clinical need. Such conditions include neuropathic neuralgia post herpatic infection, diabetic neuropathy and other areas. To date there are no new drugs for these conditions and they are often treated using antidepressants or other off label drugs such as gabapentin or pregabalin.

The use of the NSRI antidepressant milnacipran has been described in US patent application 20040147614. This invention describes the utility of drugs combining noradrenaline and serotonin reuptake inhibition in the treatment of neuropathic pain wherein the noradrenaline reuptake is more potent than the serotonin reuptake.

Tricyclic antidepressants are used in clinical practise in the treatment of neuropathic pain but such drugs generally are constrained due to their narrow therapeutic index. To date little information is available on the optimum choice of TCAs or between those which like imipramine are mixed NA and 5HT reuptake inhibitors and those like desipramine in which NA reuptake is more potent than 5HT reuptake. A number of studies of post-herpatic neuralgia and painful diabetic neuropathy have employed TCA's in mean daily doses ranging from 1002-250mg. However the results of investigations to determine drug concentrations needed for pain relief remain contradictory (Michael Clark. Chronic Pain, Depression and Antidepressants: issues and relationships, John Hopkins website reference; on the world wide web at Hopkins-arthritis.son.jhmi.edu/mngmnt/depression.html).

The use of a tricyclic antidepressant with almost total selectivity for NA reuptake in the treatment of depression with pain, depression secondary to pain or in neuropathic pain has not been described. The use of drugs which combine noradrenaline reuptake with dopamine antagonism has not been described previously and in fact there is evidence that such drugs are ineffective. The drug buproprion which combines noradrenaline and dopamine reuptake inhibition has been tested in a study of neuropathic pain but has not been found to be effective.

Despite these studies, there remains a need for improved methods of treatment of depression with painful symptoms, depression secondary to pain, and neuropathic pain.

### BRIEF SUMMARY OF THE INVENTION

We have found surprisingly that certain drugs which are noradrenaline reuptake inhibitors and also active at dopamine receptors are highly effective in treating the condition of pain predominant depression. Without being bound to any one mechanism of action such drugs relieve both physical and affective symptoms in a particularly effective way in comparison to SSRI drugs and are comparable in efficacy to SNRI drugs.

In the preferred embodiment such a drug is lofepramine. Such effectiveness however is not confined to drugs with similar chemical structure but also extends to any other drugs with comparable pharmacological action.

We have found surprisingly that lofepramine is effective in relieving the pain symptoms of depression (as distinct from other somatic symptoms of depression) independently of its effect in relieving the psychological symptoms. Lofepramine has a near term benefit in greatly relieving such painful symptoms although its effect on psychological symptoms is no more rapid than other antidepressants.

Thus in one embodiment the invention comprises compositions and methods for the treatment of depressed patients presenting with painful symptoms which comprises the administration of an effective dose of lofepramine at the dose ranges described herein.

Lofepramine is a highly safe drug and hence methods and compositions are described herein for employing lofepramine for the purposes of treating pain predominant depression at dose ranges substantially higher than has previously been used in the treatment of depression using lofepramine.

In another aspect, the invention also comprises compositions and methods for the treatment of a sub group of depressed patients namely those patients who develop depression secondary to chronic or neuropathic pain. No therapy specifically developed for this group has yet been approved.

The invention comprises compositions and methods for the treatment of depression secondary to pain which employ safe dose levels above those ranges which have previously been described or approved.

In another aspect, the invention also comprises methods and compositions for the treatment of chronic or neuropathic pain in patients who are not depressed.

Thus in one embodiment of the invention an effective dose of lofepramine in the ranges specified herein is used as a therapy to treat neuropathic pain comprising inter alia post-herpatic neuralgia, diabetic neuropathy and chemotherapy-induced neuropathy.

These and other aspects will become apparent to those of skill in the art upon reading the present disclosure.

### DETAILED DESCRIPTION OF THE INVENTION

To date, no evidence has been developed which shows the utility of drugs which combine primary NA reuptake with dopamine antagonism.

In accordance with the present invention, it has been surprisingly found that a drug embodying such pharmacology is effective in the treatment of depression with somatization referred to here as “pain predominant type depression”, in the treatment of depression secondary to chronic or neuropathic pain, and in neuropathic pain itself. In the preferred embodiment of the invention, the drug is lofepramine.

There are a number of ways in which the ratio of NA to 5HT reuptake in antidepressant molecules may be measured. The selectivity a drug for various receptors may be compared by use of comparative Ki's and not comparative IC 50 values. (Bolden-Watson, C and Richelson, E., Life Sci, (52), 1993, 1023-1029), which is hereby incorporated by reference. For lofepramine the relative potencies of NA: 5HT based on Ki data are 1,200:1

Thus the ratio of NA to 5HT as measured by Ki for lofepramine is over 1000:1. Lofepramine is therefore not a SNRI or NSRI as usually measured, but is predominantly a NA reuptake inhibitor. The second pharmacological action of lofepramine of note is that while it is not a dopamine reuptake inhibitor it is a dopamine antagonist.

In accordance with certain aspects of the present invention, the dopaminergic activity of lofepramine as a valuable additional property in the treatment of a variety of conditions and disorders. Lofepramine has been shown to have potent activity at the D2 receptor. Its effectiveness in the diseases which are the subject of this invention is surprising given the ineffectiveness of buproprion.

Significantly, the use of lofepramine, and compounds with the pharmacological profile described herein, provides a suitable manner to overcome safety concerns associated with use of highly effective but toxic TCAs known in the art, such as desipramine. In accordance with the present invention, it has been unexpectedly found that lofepramine has a very low cardiotoxicity, although its main metabolite is in fact desipramine. An analysis of fatal poisoning by antidepressants in Scotland, England and Wales was carried out by Buckley and Mcmanus (2002). Their data showed that for the tricyclic antidepressants as a class fatalities per million prescriptions was 34.8; for desipramine it was 200.9; for the leading antidepressant venlafaxine it was 13.2 and for the SSRI class it was only 1.6. SSRI;s are regarded by physicians as very safe drugs (which is their main advantage over TCA's). However the data for lofepramine shows a fatality rate from overdose of 1.3 per million prescriptions indicating that lofepramine is safer than the average for the SSRI class.

Arrythmia is the most serious consequence of TCA overdose. Progression of ECG changes are relatively predictable and related to the severity of the overdose. Mild oversose produces sinus tachycardia, mostly as a result of anticholinergic effects. More severe overdoses result in prolonged QRS and QTc intervals, followed by a prolonged PR interval, and finally ventricular arrhythmias. Sjogren (1987) has demonstrated that tricyclic antidepressants such as amitriptyline, imipramine and desipramine prolong the ECG interval in rats infused with these antidepressants. Lofepramine on the other hand does not, and its effect is similar to that of the control vehicle.

As such, it has been unexpectedly found that lofepramine, optionally administered alone in the absence of any neurotransmitter precursor compounds, provides a highly effective and well tolerated treatment for pain predominant type depression, in the treatment of depression secondary to chronic or neuropathic pain, and in neuropathic pain itself. In the preferred embodiment of the invention, the drug is lofepramine.

### D. Compounds of the Invention

The preferred compounds of the present invention include lofepramine and its pharmaceutically acceptable salts, i.e., hydrochloride salt (the active ingredient in Gamanil™ and Lomont™). The chemical structure of lofepramine is shown below.

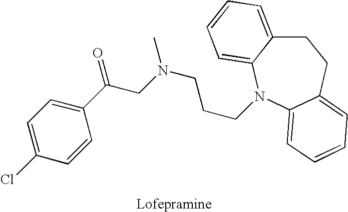

Lofepramine is a tricyclic antidepressant approved in a number of European countries including the UK and Ireland. Lofepramine is structurally similar to imipramine and is extensively metabolized to desipramine. It is believed that its antidepressant activity stems from the facilitation of noradrenergic neurotransmission by uptake inhibition, and possibly by the additional facilitation of serotoninergic neurotransmission. The overall therapeutic efficacy of lofepramine is comparable to that of imipramine, amitriptyline, clomipramine, maprotilene (maprotiline), and mianserin in patients with depression of varying severity and coexisting anxiety.

More particularly, lofepramine is a TCA that possesses a high NE (sometimes referred to as NA): 5-HT ratio and possesses stimulatory effects of 5-HT synthesis. Lofepramine also possesses dopaminergic effects and, very importantly, has very low cardiotoxicity. Additionally, it is noted that lofepramine possesses a NE: 5-HT ratio that is higher than that of milnacipran.

As mentioned above, lofepramine acts primarily as a NE reuptake inhibitor, although it also has 5-HT reuptake effects. In one study, lofepramine's NE IC50 was found to be 4 times that of its 5-HT IC50 (Segawa et al 1977). In a more accurate comparison of the relative potencies at NA and 5HT, Bolden-Watson showed that lofepramine has a NA over 5HT selectivity of 1,200:1. However, in accordance with the present invention, lofepramine has also been found to exert additional pharmacological properties. For instance, lofepramine has been shown to up-regulate serotonin synthesis in the brain. Lofepramine has also been shown to have a very low cardiotoxicity, with toxic levels similar to that found in the SSRI's. Further, lofepramine has been found to exert its effects on dopamine D2 receptors. Unlike a number of other tricyclic antidepressants lofepramine does not induce sedation. While any compound which possesses properties similar to lofepramine in these respects may be useful in the present invention, lofepramine and its pharmaceutically acceptable salts are the preferred compound of the invention

Also falling within the scope of the present invention are the in vivo metabolic products of the lofepramine compounds described herein, including desipramine. Such products may result, for example, from the oxidation, reduction, hydrolysis, amidation, esterification, and the like of the administered compound, primarily due to the enzymatic processes. Accordingly, the invention includes compounds produced by a process comprising contacting a lofepramine compound of the invention with a mammalian tissue or a mammal for a period of time sufficient to yield a metabolic product thereof. Such products typically are identified by preparing a radio-labeled (e.g. C¹⁴ or H³) lofepramine compound of the invention, administering it in a detectable dose (e.g., greater than about 0.5 mg/kg) to a mammal such as a rat, mouse, guinea pig, monkey, or human, allowing sufficient time for metabolism to occur (typically about 30 seconds to 30 hours), and isolating its conversion products from urine, blood, tumor, or other biological samples. These products are easily isolated since they are labeled (others are isolated by the use of antibodies capable of binding epitopes surviving in the metabolite). The metabolite structures are determined in conventional fashion, e.g., by MS or NMR analysis. In general, analysis of metabolites may be done in the same way as conventional drug metabolism studies well-known to those skilled in the art. The conversion products, so long as they are not otherwise found in vivo, are useful in diagnostic assays for therapeutic dosing of the compounds of the invention, even if they possess no biological activity of their own.

The main metabolite of lofepramine is the tricyclic drug desipramine. Desipramine may contribute to the pharmacological actions of lofepramine but lofepramine is not a pro-drug for desipramine and lofepramine has a of NA:5HT ratio of about four times that of desipramine. However, the non-toxicity of lofepramine in overdose appears not be fully understood since it would be expected that in such cases significant plasma levels of desipramine would be generated.

The metabolites of lofepramine include three compounds that are also common to the metabolism of imipramine, namely, desipramine, 2-hydroxydesipraminne, and didesmethylimipramine. Lofepramine also generates three unique metabolites of which two have been identified as 2-hydroxyllofepramine and desmethyllofepramine (Strangarden, K and P. O. Gunnarsson. 1994. Metabolism of lofepramine and imipramine in liver microsomes from rat and man. Xenobiotica, 24, No. 8, 703-711), which is hereby incorporated by reference.

In another aspect of the invention, it is believed that the unique metabolites of lofepramine act in a cardioprotective manner to counter the toxic effects of the desipramine metabolite. If so, then these metabolites, depending on their pharmacokinetics, may also be safe and effective drugs for the treatment of the conditions and disorders described herein. In a highly preferred embodiment of the present invention, 2-hydroxyllofepramine and desmethyllofepramine are compounds of the present invention, either individually or in combination or in the ratios in which they occur following metabolism of lofepramine.

### E. Methods of the Invention

In accordance with the present invention, it has been discovered that compounds exhibiting activity as a potent noradrenaline reuptake inhibitor (e.g., a NA: 5HT ratio of greater than or equal to about 1000:1), and activity at the dopamine D2 receptor sites (e.g., lofepramine) are effective in the treatment and prevention of various diseases and disorders associated with noradrenaline reuptake, such as pain predominant-type depression, depression secondary to chronic or neuropathic pain, and neuropathic pain itself. In the preferred embodiment of the invention, the drug is lofepramine.

In certain aspects of the present invention, it has been found that lofepramine's action on pain in depression, e.g., pain predominant-type depression or depression secondary to chronic or neuropathic pain, is independent of its action on the psychological symptoms as measured by the Hamilton Rating Scale for Depression (HAM-D). Lofepramine has a highly effective and early impact on pain symptoms, such as back pain, chest pain, headaches, muscle pain and non-specific pains. For patients who present with pain predominant-type depression or depression secondary to chronic or neuropathic pain, the early relief of such symptoms is helpful and reinforces belief that the drug is having a beneficial effect. Without being bound to any mechanism of action it appears that pain relief contributes to improved cognitive and emotional response, which in turn assists in the relief of the depression. The delay in the onset of antidepressant action (up to 4 weeks) is one of the major drawbacks of antidepressant therapy. For patients who do not present with pain co-morbid with depression, this early signal of getting better will however not be available.

As such, one aspect of the invention relates to methods for treating pain predominant-type depression, depression secondary to chronic or neuropathic pain, or neuropathic pain itself, in a subject in need thereof, comprising administering to the patient a composition comprising a therapeutically effective amount of lofepramine or a pharmaceutically acceptable salt thereof.

While the use of lofepramine in the treatment of depression is generally known, it was unexpectedly discovered in accordance with the present invention that lofepramine is particularly effective in the treatment of depression with painful symptoms or “somatizations” of depression. Such conditions should not be confused with fibromyalgia, as the diagnosis of the latter excludes major depression. As such, in one embodiment of the invention, lofepramine may be employed as the single active ingredient of a medicament for the treatment of “pain predominate-type depression”, i.e., the composition consists essentially of lofepramine or a pharmaceutically acceptable salt thereof.

In other aspects, lofepramine has been found to be effective in relieving neuropathic pain itself. Such pain may include diabetic neuropathy, chemotherapy induced neuropathy, postherpatic neuralgia, and other related conditions.

In another aspect of the invention, due to the unexpected discovery of the cardioprotective aspects of the compounds of the present invention, the methods of the invention may include identification of a subject at risk of an adverse cardiac event. As such, in another aspect of the invention, a method for treating or preventing depression in a subject in need thereof is provided, comprising administering to the patient a composition comprising a therapeutically effective amount of lofepramine or a pharmaceutically acceptable salt thereof, wherein the subject is at risk of an adverse cardiac event. In certain embodiments, the method comprises identifying the subject as being at risk of an adverse cardiac event. Adverse cardiac events include any cardiac event generally recognized by those skilled in the art, including myocardial infarction, congestive heart failure, irregular heat beat, stroke, etc.

In one embodiment of the present invention, lofepramine in immediate release form is administered to the subject, e.g., a pediatric subject, more than once a day. The first dose is in the morning, such that the dose and half-life of the drug are sufficient to provide effective treatment during school or work hours.

In one embodiment of the present invention, lofepramine in immediate release form, but at a higher dose than in the previous embodiment, is administered once a day in the morning. Given the safety of lofepramine, once a day dosing can be achieved for the purposes herein without recourse to any sustained release technologies.

In a preferred embodiment lofepramine may be administered in a extended release once a day format using techniques known in the art. The once a day form of lofepramine will provide additional benefits in further reducing the already mild side effects of the immediate release form and also have the convenience of once a day dosing.

According to the methods of the invention, the compound(s) may be administered to the subject via any drug delivery route known in the art. Specific exemplary administration routes include peripheral and central routes such as oral, ocular, rectal, buccal, topical, nasal, ophthalmic, subcutaneous, intramuscular, intraveneous (bolus and infusion), intracerebral, transdermal, and pulmonary. In a preferred embodiment, the composition is administered orally via tablet.

The term “therapeutically effective amount”, as used herein, refers to an amount of a pharmaceutical agent to treat, ameliorate, or prevent the identified disease or condition, or to exhibit a detectable therapeutic or inhibitory effect. The effect can be detected by any means known in the art. The precise effective amount for a subject will depend upon the subject's body weight, size, and health; the nature and extent of the condition; and the therapeutic or combination of therapeutics selected for administration. Therapeutically effective amounts for a given situation can be determined by routine experimentation that is within the skill and judgment of the clinician.

More particularly, preferred therapeutically effective amounts of the compound(s) of the present invention include administration at doses that vary from 40 mg to 420 mg, administered in single or divided doses, depending upon the route of administration and the age and size of the subject, as recognized by those skilled in the art. Guidance as to particular dosages and methods of delivery is provided in the literature and is generally available to practitioners in the art.

Recommended dosages for lofepramine as employed in practice the present invention are 70 mg twice daily (140 mg), or up to three times per day (210 mg) depending on patient response. Lofepramine may also be employed up to doses of 420 mg per day given its low cardiotoxicity. Thus, in one embodiment, doses of lofepramine range from about 70 mg to about 140 mg per day, about 140 mg to about 210 mg per day, or about 210 mg to about 420 mg per day. In other embodiments, the dose may range from about 50 mg per day to about 140 mg per day, from about 210 mg per day to about 280 mg per day, or from about 280 mg per day to about 400 mg per day.

Higher doses of lofepramine may be employed for shorter periods (such as one to two weeks) in order to obtain immediate or short term relief from painful symptoms, with a titration down to lower doses if desired. In certain embodiments, the use of high doses of lofepramine in the range of 280-400 mg per day for the first two weeks to effectively relieve pain, followed by a titration down to lower doses for the continuing treatment of depression and relief of painful symptoms may be effective in treating patients presenting with pain predominant-type depression or depression secondary to chronic or neuropathic pain. In treating neuropathic pain itself, in certain embodiments, the starting dose may be in the range of 210-400 mg for an initial period, followed by a titration down to lower doses.

The exact dosage will be determined by the practitioner, in light of factors related to the subject that requires treatment and the form of lofepramine used (e.g., the salt form). Dosage and administration are adjusted to provide sufficient levels of the active agent(s) or to maintain the desired effect. Other factors which may be taken into account include the severity of the disease state, general health of the subject, age and weight of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation.

### F. Pharmaceutical Compositions of the Invention

In yet another aspect of the present invention, pharmaceutical compositions useful in the methods of the invention are provided. The pharmaceutical compositions of the invention may be formulated with pharmaceutically acceptable excipients such as carriers, solvents, stabilizers, adjuvants, diluents, etc., depending upon the particular mode of administration and dosage form. The pharmaceutical compositions should generally be formulated to achieve a physiologically compatible pH, and may range from a pH of about 3 to a pH of about 11, preferably about pH 3 to about pH 7, depending on the formulation and route of administration. In alternative embodiments, it may be preferred that the pH is adjusted to a range from about pH 5.0 to about pH 8.0.

In a particularly preferred embodiment of the present invention, the pharmaceutical compositions of the invention comprise a therapeutically effective amount of lofepramine or a pharmaceutically acceptable salt thereof, together with one or more pharmaceutically acceptable excipients. For instance, when the pharmaceutical composition is formulated as an oral tablet, the composition preferably comprises from about 0.1 mg to about 70 mg of the lofepramine compound, more preferably from about 5 mg to about 100 mg. As discussed above, the exact amount of lofepramine may vary. In another preferred embodiment, the pharmaceutical composition is entirely free from amino acids such as phenylalanine. In another preferred embodiment, the pharmaceutical composition comprises the lofepramine compound as its only active ingredient, i.e., there are no other active ingredients included in the pharmaceutical composition.

In a more particularly preferred embodiment of the present invention, the pharmaceutical compositions of the invention comprise a tablet, capsule, lozenge or other orally available drug which comprises a single dose of lofepramine or a pharmaceutically acceptable salt suitable to provide effective once a day therapy for the conditions herein.

In an alternative embodiment of the present invention, the pharmaceutical compositions of the invention may comprise a combination of active ingredients, including but not limited to a second therapeutic agent useful in the treatment of pain predominant type depression, depression secondary to chronic or neuropathic pain, or neuropathic pain itself. Therapeutic amounts of second agents are generally known in the art or may be determined by the skilled clinician.

Formulations of the present invention, e.g., for parenteral or oral administration, are most typically solids, liquid solutions, emulsions or suspensions, while inhaleable formulations for pulmonary administration are generally liquids or powders, with powder formulations being generally preferred.

The term “pharmaceutically acceptable excipient” refers to an excipient for administration of a pharmaceutical agent, such as the compounds of the present invention. The term refers to any pharmaceutical excipient that may be administered without undue toxicity. Pharmaceutically acceptable excipients are determined in part by the particular composition being administered, as well as by the particular method used to administer the composition. Accordingly, there exists a wide variety of suitable formulations of pharmaceutical compositions of the present invention (see, e.g., Remington's Pharmaceutical Sciences).

Suitable excipients may be carrier molecules that include large, slowly metabolized macromolecules such as proteins, polysaccharides, polylactic acids, polyglycolic acids, polymeric amino acids, amino acid copolymers, and inactive virus particles. Other exemplary excipients include antioxidants such as ascorbic acid; chelating agents such as EDTA; carbohydrates such as dextrin, hydroxyalkylcellulose, hydroxyalkylmethylcellulose, stearic acid; liquids such as oils, water, saline, glycerol and ethanol; wetting or emulsifying agents; pH buffering substances; and the like. Liposomes are also included within the definition of pharmaceutically acceptable excipients.

The pharmaceutical compositions of the invention may be formulated in any form suitable for the intended method of administration. When intended for oral use for example, tablets, troches, lozenges, aqueous or oil suspensions, non-aqueous solutions, dispersible powders or granules (including micronized particles or nanoparticles), emulsions, hard or soft capsules, syrups or elixirs may be prepared. Compositions intended for oral use may be prepared according to any method known to the art for the manufacture of pharmaceutical compositions, and such compositions may contain one or more agents including sweetening agents, flavoring agents, coloring agents and preserving agents, in order to provide a palatable preparation.

Pharmaceutically acceptable excipients particularly suitable for use in conjunction with tablets include, for example, inert diluents, such as celluloses, calcium or sodium carbonate, lactose, calcium or sodium phosphate; disintegrating agents, such as croscarmellose sodium, cross-linked povidone, maize starch, or alginic acid; binding agents, such as povidone, starch, gelatin or acacia; and lubricating agents, such as magnesium stearate, stearic acid or talc. Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed. Tablets can be formulated as controlled release drugs using techniques known in the art so as to provide once a day dosing within the ranges as specified herein.

Formulations for oral use may be also presented as hard gelatin capsules where the active ingredient is mixed with an inert solid diluent, for example celluloses, lactose, calcium phosphate or kaolin, or as soft gelatin capsules wherein the active ingredient is mixed with non-aqueous or oil medium, such as glycerin, propylene glycol, polyethylene glycol, peanut oil, liquid paraffin or olive oil.

In another embodiment, pharmaceutical compositions of the invention may be formulated as suspensions comprising a compound of the present invention in admixture with at least one pharmaceutically acceptable excipient suitable for the manufacture of a suspension. In yet another embodiment, pharmaceutical compositions of the invention may be formulated as dispersible powders and granules suitable for preparation of a suspension by the addition of suitable excipients.

Excipients suitable for use in connection with suspensions include suspending agents, such as sodium carboxymethylcellulose, methylcellulose, hydroxypropyl methylcelluose, sodium alginate, polyvinylpyrrolidone, gum tragacanth, gum acacia, dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycethanol), a condensation product of ethylene oxide with a partial ester derived from a fatty acid and a hexitol anhydride (e.g., polyoxyethylene sorbitan monooleate); and thickening agents, such as carbomer, beeswax, hard paraffin or cetyl alcohol. The suspensions may also contain one or more preservatives such as acetic acid, methyl and/or n-propyl p-hydroxy-benzoate; one or more coloring agents; one or more flavoring agents; and one or more sweetening agents such as sucrose or saccharin.

The pharmaceutical compositions of the invention may also be in the form of oil-in-water emulsions. The oily phase may be a vegetable oil, such as olive oil or arachis oil, a mineral oil, such as liquid paraffin, or a mixture of these. Suitable emulsifying agents include naturally-occurring gums, such as gum acacia and gum tragacanth; naturally occurring phosphatides, such as soybean lecithin, esters or partial esters derived from fatty acids; hexitol anhydrides, such as sorbitan monooleate; and condensation products of these partial esters with ethylene oxide, such as polyoxyethylene sorbitan monooleate. The emulsion may also contain sweetening and flavoring agents. Syrups and elixirs may be formulated with sweetening agents, such as glycerol, sorbitol or sucrose. Such formulations may also contain a demulcent, a preservative, a flavoring, or a coloring agent, or a combination of these.

### G. Combination Therapy

In another aspect of the invention, it has been unexpectedly discovered that lofepramine may be combined with another active ingredient to treat pain predominant type depression, depression secondary to chronic or neuropathic pain, or neuropathic pain itself. More particularly, SNRI and NSRI drugs which are single molecules cannot vary the ratio of NE: 5-HT activity. However, in accordance with the invention, such variation has been found to be desirable. As such, another aspect of the invention relates to the combination of a SNRI or NSRI (i.e., a primary NE reuptake inhibitor) with a primary 5-HT reuptake inhibitor. In a preferred embodiment, the NE: 5-HT ratio employed is greater than 1:1, more preferably in the range about 2-10:1, and even more preferably between about 10-100:1.

Thus, in another embodiment of the invention, lofepramine, which is primarily a NE reuptake inhibitor, is combined with a compound which is primarily a 5-HT reuptake inhibitor. In a preferred embodiment, lofepramine is combined with citalopram. However, the skilled artisan will recognize that a variety of active ingredients may be administered in combination with the primary NE reuptake inhibitor that may act to augment or synergistically enhance the activity of the primary NE reuptake inhibitor (e.g., lofepramine). Therapeutic doses may be determined by one of ordinary skill in the art. In a particularly preferred embodiment of the invention, 5 mg of citalopram and 100 mg of lofepramine are administered daily.

The primary NE reuptake inhibitor (e.g., lofepramine) and the primary 5-HT reuptake inhibitor (e.g., citalopram) may be combined in any manner known in the art such as a unitary dosage form, or in separate dosage forms intended for simultaneous or sequential administration to a patient in need of treatment. When administered sequentially, the combination may be administered in two or more administrations. In an alternative embodiment, it is possible to administer one or more compounds of the present invention and one or more additional active ingredients by different routes.

According to the methods of the invention, the combination of active ingredients may be: (1) co-formulated and administered or delivered simultaneously in a combined formulation; (2) delivered by alternation or in parallel as separate formulations; or (3) by any other combination therapy regimen known in the art. When delivered in alternation therapy, the methods of the invention may comprise administering or delivering the active ingredients sequentially, e.g., in separate solution, emulsion, suspension, tablets, pills or capsules, or by different injections in separate syringes. In general, during alternation therapy, an effective dosage of each active ingredient is administered sequentially, i.e., serially, whereas in simultaneous therapy, effective dosages of two or more active ingredients are administered together. Various sequences of intermittent combination therapy may also be used.

To assist in understanding the present invention, the following Examples are included. The experiments relating to this invention should not, of course, be construed as specifically limiting the invention and such variations of the invention, now known or later developed, which would be within the purview of one skilled in the art are considered to fall within the scope of the invention as described herein and hereinafter claimed.

### EXAMPLES

The present invention is described in more detail with reference to the following non-limiting examples, which are offered to more fully illustrate the invention, but are not to be construed as limiting the scope thereof. The examples illustrate the preparation of certain compounds of the invention, and the testing of these compounds. Those of skill in the art will understand that the techniques described in these examples represent techniques described by the inventors to function well in the practice of the invention, and as such constitute preferred modes for the practice thereof. However, it should be appreciated that those of skill in the art should in light of the present disclosure, appreciate that many changes can be made in the specific methods that are disclosed and still obtain a like or similar result without departing from the spirit and scope of the invention.

### Example 1

Three patients who fulfilled criteria for major depression present with multiple aches and pains for which there is no obvious physical cause. Each is commenced on lofepramine 70 mg twice daily. All three show improvement in all symptoms including pain within four weeks. The final dose of medication ranges from 140mg daily to 280mg daily.

### Example 2

Four patients at a diabetic clinic present with severe neuropathic pain. Conventional analgesics are of no benefit. They are subsequently prescribed duloxetine and also failed to respond. They are then prescribed lofepramine 70 mg tds, which produces a dramatic relief of pain in 3 of the 4 cases. Escalation of the dose in patient 4 subsequently results in improvement.

1. A method for the treatment of depression in which pain is a predominant presenting feature comprising the administration to a patient in need of such therapy a medicament comprising an effective dose of lofepramine.

2. A method of claim 1 wherein the dose of lofepramine is between 50 mg per day and 400 mg per day.

3. A method for the treatment of depression secondary to chronic pain or neuropathic pain comprising the administration to a patient in need of such therapy a medicament comprising an effective dose of lofepramine.

4. A method of claim 3 wherein the dose of lofepramine is between 50 mg per day and 400 mg per day.

5. A method for the treatment of neuropathic pain including post herpatic neuralgia, diabetic neuropathy, chemotherapy-induced neuropathy and related conditions comprising the administration to a patient in need of such therapy of a medicament comprising an effective dose of lofepramine.

6. A method of claim 5 wherein the dose of lofepramine is between 50 mg per day and 400 mg per day.

7-9. (canceled)

10. The method according to claim 1, wherein said medicament is essentially free of amino acids.

11. The method according to claim 1, wherein said medicament further comprises a second component which is primarily a 5-HT reuptake inhibitor.

12. The method according to claim 11, wherein the 5-HT reuptake inhibitor is citalopram.

13. The method according to claim 1, wherein the patient is at risk of an adverse cardiac event.

14. The method according to claim 3, wherein said medicament is essentially free of amino acids.

15. The method according to claim 3, wherein said medicament further comprises a second component which is primarily a 5-HT reuptake inhibitor.

16. The method according to claim 15, wherein the 5-HT reuptake inhibitor is citalopram.

17. The method according to claim 3, wherein the patient is at risk of an adverse cardiac event.

18. The method according to claim 5, wherein said medicament is essentially free of amino acids.

19. The method according to claim 5, wherein said medicament further comprises a second component which is primarily a 5-HT reuptake inhibitor.

20. The method according to claim 19, wherein the 5-HT reuptake inhibitor is citalopram.

21. The method according to claim 5, wherein the patient is at risk of an adverse cardiac event.
